(19) United States
(12) Patent Application Publication
(10) Pub. No.: US 2005/0129780 A1
(43) Pub. Date: Jun. 16, 2005

(54) Relief of aids symptoms

(76) Inventors: Terri Lee Holcomb-Halstead, Grand Terrace, CA (US); Zhenguo Wang, Grand Terrace, CA (US)

Correspondence Address: ROBERT D. FISH;RUTAN & TUCKER LLP, 611 ANTON BLVD 14TH FLOOR, COSTA MESA, CA, 92626-1931, US

(21) Appl. No.: 10/515,773

(22) PCT Filed: May 29, 2003
(86) PCT No.: PCT/US03/17131

(30) Foreign Application Priority Data
May 29, 2002 (US) 10159723
May 29, 2002 (US) 10159434
May 29, 2002 (US) 10159417
May 29, 2002 (US) 10159433
May 29, 2002 (US) 10159747
Jul. 10, 2002 (US) 60395227

(51) Int. Cl. A61K 35/78; A61K 35/38

(52) U.S. Cl. 424/551; 424/728; 424/773; 424/757

(57) ABSTRACT

Compositions and methods of improving the quality of life of an AIDS patient comprise combinations of herbal compositions and at least one of a bile product and a chelator. Additional subject matter includes methods of marketing compositions and methods.

This application claims the benefit of U.S. utility er 10/159,723 filed on May 29, 2003, U.S. utility application Ser. No. 10/159,434 filed on May 29, 2003, U.S. utility application Ser. No. 10/159,417 filed on May 29, 2003, U.S. utility application Ser. No. 10/159,433 filed on May 29, 2003, U.S. utility application Ser. No. 10/159,747 and U.S. provisional application No. 60/395,227 filed on Jul. 10, 2002 all incorporated herein by reference in their entirety.

### FIELD OF THE INVENTION

The field of the invention is compositions to relieve AIDS (Acquired Immuno Deficiency Syndrome) symptoms.

### BACKGROUND

Most patients infected with the HIV virus will develop AIDS, reflecting a breakdown in their immune system's capability to ward off foreign and “self”-generated antigens. Viral infections are unfortunately an almost unavoidable challenge to the health of most human and other mammals, and while many viral infections are successfully cleared by the immune system of the infected individual before substantial damage arises, some viral infections lead to severe damage or even death. There are many known antiviral drugs, however, all or almost all of them suffer from one or more disadvantages, most notably adverse side-effects, built-up of viral resistance, complicated administration schedules, and often high cost. Therefore, there is a need for simple and effective antiviral compositions that are well tolerated, simple to administer, and relatively inexpensive.

Unless expressly stated otherwise, the term “AIDS” should be interpreted herein to include ARC (AIDS related complex). Additionally, patients who develop AIDS often experience various uncomfortable, and often painful, symptoms. Although some compositions are available that are marketed toward relieving some of the symptoms experienced by AIDS patients, there is still a need for improved and lower cost compositions for the relief of AIDS symptoms.

### SUMMARY

The present subject matter is directed to compositions and methods of improving the quality of life of an ADS patient. As used herein, “quality of life” means sleep, energy level, discomfort, appetite, energy level, and mental clarity. Particularly contemplated compositions include herbal extracts prepared from plants selected from the group consisting of: Radix Ginseng; Radix Angelicae Sinensis; Radix Astragali seu Hedysari; Fructus Ligustri Lucidi; Radix Sophorae Flavescentis; Radix Trichosanthis; Herba Agrimoniae; Ganoderma Lucidum seu Japonicum; Radix Rehmanniae; Cordyceps; Bomeolum Syntheticum; and calculus Bovis.

Additionally, contemplated subject matter includes methods of marketing compositions to alleviate symptoms associated with a disease, such as AIDS.

Various objects, features, aspects and advantages of the present invention will become more apparent from the following detailed description of preferred embodiments of the invention, along with the accompanying drawings in which like numerals represent like components.

### DETAILED DESCRIPTION

The inventors discovered that a combination of any of several herbal compositions (or their constituents) in combination with at least one of a chelator and a bile product, are effective in improving quality of life for AIDS patients.

An “herbal composition” as used herein means the whole or any part of a plant including a root, leaf, stem and the flower that is used or processed, including extracts thereof. For example, an herbal composition may be obtained through any of the following procedures: extraction; emulsion, grinding, etc. The herb itself may be administered in any kind of form. In most cases, the herb is administered in an oral form, but may also be parenterally administered. For example, acetyl salycillic acid in pill form is aspirin, but if extracted, is an herb.

General Information

The inventors discovered that symptoms of a viral infection, especially HIV, can be significantly improved by coadministration of an antiviral agent with a chelator. More specifically, the inventors contemplate that particularly suitable chelators deplete the viral environment sufficiently to promote disintegration of the viral envelope.

It is further contemplated that suitable compositions will reduce the viral serum titer of a virus in an amount of at least 10% (e.g., as determined by RT-PCR), and especially contemplated viruses include retroviruses (e.g., HIV, HCV), dsDNA and ssDNA viruses.

With respect to the administration of contemplated compositions, it should be recognized that various protocols are suitable, and especially contemplated protocols include substantially simultaneous administration of the chelator (e.g., coadministration in a single tablet), or administration of the chelator (or antiviral agent) while there is a measurable concentration of the antiviral agent (or chelator) in the patient. For example, it is contemplated that suitable antiviral agents may be orally administered, while the chelator is parenterally administered (e.g., via injection or mucosal presentation).

Consequently, the dosage and formulation of contemplated antiviral agents and chelators may vary substantially, however, it is preferred that the antiviral agent is administered in approved and/or known dosages and formulations. Similarly, it is preferred that dosages and formulations of appropriate chelators are identical or similar to those known in the art.

The inventors further contemplate that treatment of a symptoms of a viral infection can be significantly improved by administration of an antiviral agent in a time-release formulation. More specifically, the inventors contemplate that a reverse transcriptase inhibitor in a time-release formulation is administered to a patient suffering from a viral infection. Particularly contemplated viruses include retroviruses (e.g., HIV, HCV), ssDNA and dsDNA.

The inventors recognized that various herbal compositions exhibit significant antiviral activity and that reverse transcriptase inhibitors (RTI) may be isolated for such herbal compositions. Moreover, the inventors contemplate that such RTIs can be characterized and/or synthesized de novo.

With respect to the identification of an RTI in contemplated plants, it should be appreciated that numerous assays are known in the art, and can readily be adapted to a screening process in which a fractions of a herbal composition are screened for RTI activity. For example, U.S. Pat. No. 6,130,036 to Loeb et al. describes a high throughput assay system in which positive selective pressure is employed to select and/or identify an RTI. Once a fraction has been identified as having RTI activity, it is contemplated that further separation of the components in that fraction will eventually lead to an isolated (single or complex) compound.

It is still further contemplated that such isolated compounds may then be characterized using various forms of mass spectroscopy (e.g., ESMS, FAB-MS, GC-MS, etc.), UV-, IR-, and VIS-spectroscopy, atom absorption spectroscopy, various forms of NMR (¹H-NMR, ¹³C-NMR, NOE-NMR, etc.), or other analytical method. While not limiting to the inventive subject matter, it is preferred that such characterization methods will lead to a chemical structure of the RTI, which may be employed to synthesize the RTI de novo, or to modify the structure to arrive at an RTI with improved or altered physico-chemical properties.

Particularly contemplated modifications of isolated and characterized RTIs include increased specificity towards the viral polymerase over non-specific interactions with non-reverse transcript-ase molecules in a cell or biological system, higher affinity of the modified RTI towards the reverse transcriptase, reduced toxicity, increased solubility, etc.

Consequently, it is contemplated that pharmacological composition comprises a synthetic reverse transcriptase inhibitor having a structure of a molecule that is present in a herbal composition demonstrated to have an antiviral effect, wherein the molecule produces at least in part of the antiviral effect.

With respect to the dosage of contemplated compositions, it is contemplated that the RTI is present in a single dose in a concentration such that the viral titer is reduced at least 20% over a period of at least 6 hours, more at least 30% over a period of at least 8 hours, and most preferably at least 45% over a period of at least 12 hours. The chelating agent is advantageously administered in a dosage such that the serum Mg²⁺ and/or Ca²⁺ concentration is reduced at least 20% over a period of at least 6 hours, more preferably at least 35% over a period of at least 12 hours, and most preferably at least 45% over a period of at least 12 hours.

As used herein, the term “administer” or “administered” means that a person (whether a patient or not) is taking the composition on his or her own or under the guidance of a health care professional. With respect to the administration of contemplated compositions, it should be recognized that various protocols are suitable, and especially contemplated protocols include oral, topical, and parenteral administration. Consequently, the formulation of contemplated compositions may vary substantially, however, it is preferred that the RTI is administered in approved and/or known formulations.

Furthermore, the term “HIV related condition” as used herein refers to intrinsic and extrinsic challenges to an immune system that may develop into an apparent (i.e., detectable by diagnotic tools) disease while the patient has a detectable HIV serum virus titer. Particularly contemplated conditions include bacterial infections (e.g., pneumocystis camii, tuberculosis, salmonellosis, mycobacterium avium complex, etc.), viral infections (e.g., cytomegalovirus, herpes simplex, hepatitis, varicella zoster, Epstein-barr, etc.), fungal infections (e.g., candidiasis, cryptococcal meningitis, histoplasmosis, etc.), parasite infections (e.g., toxoplasmosis, cryptosporidiosis, etc.), and Kaposi sarcoma.

Suitable compositions are described in copending provisional patent applications with the title “Treatment of Virus Using Chelator and Antiviral Agent” by Bruce Halstead et al., filed on or about May 30, 2001, “Time Release Chelators” by Bruce Halstead et al., filed on or about May 30, 2001, and “Time Release reverse transcriptase inhibitors” by Bruce Halstead et al., filed on or about May 30, 2001, all of which are incorporated by reference herein.

In a preferred aspect of the inventive subject matter, a method of treating a patient comprises one step in which a patient infected with HIV is diagnosed with an HIV related condition. In a further step, a composition is administered to the patient that comprises at least one of a chelator and an antiviral agent, wherein the antiviral agent comprises a herbal composition or a synthetic or isolated compound from a plant that is demonstrated to have an antiviral effect. It should be recognized that all patients infected with an HIV virus may be treated using contemplated methods, however, patients with a CD4⁺ count of less than 200 are particularly contemplated. Consequently, especially preferred patients include patients with developing or fully developed AIDS, wherein such patients may or may not receive pharmacological treatment.

With respect to the administration of contemplated compounds, it should be appreciated that a particular dosage and regimen will typically depend on the particular HIV-related condition. It is generally contemplated that the dosage, route and formulation is substantially identical or similar to those described in the copending provisional applications. However, where appropriate, alternative dosages, routes, and formulations may be employed, and in fact all dosages formulations and routes are contemplated that result in a positive response of the patient to the administration.

Herbal Compositions

Several classes of herbal compositions are contemplated:

Herbal Composition #1

The inventors contemplate that herbal compositions can act as antiviral agents. Such antiviral agents particularly include direct antiviral agents and indirect antiviral agents. As used herein, the term “direct antiviral drug” refers to an agent that directly interferes with one or more viral components. For example, virus protein specific antibodies, reverse transcriptase inhibitors or protease inhibitors are considered direct antiviral agents, because such compounds directly bind and to and/or reduce the activity of their respective viral target structures. As also used herein, the term “indirect antiviral drug” refers to a compound that indirectly interferes with a replication or propagation of a virus, and particularly include immunomodulatory agents (e.g., cytokines, various nucleoside analogs, and/or Zn²⁺). However, it should be appreciated that chelators are explicitly excluded from the definitions of direct and indirect antiviral compounds.

Especially preferred antiviral compounds include herbal compositions and/or one or more isolated compounds (isolated from the plant or synthesized de novo) that are present in a herbal composition demonstrated to have an antiviral effect. Particularly suitable plants for contemplated extracts and isolated compounds include Abies webbiana; Acacia spec. Acacia Arabia; Agrimonia eupatoria; Ajuga decumbens; Allium cepa; Allium sativum; Aloe vera; Alternanthera philoxeroides or sessiles; Ammi maius; Andographis paniculata; Apium graveolens; Apium leptophyllum; Arachis hypogaea; Arctium lappa; Amebia euhcroma; Asparagus racemosus; Astragalus spinosus; Astragalus lentingosis swainsonine; Buchenavia capita; Bryonia cretica ssp. Dioica; Bryonia angustifolia; Camellia theifera; Camellia sinensis; Cedrela toona; Chrysanthemum morifolium; Coffea arabica; Coptis chinesis; Coptis teetoides; Coptis japonica; Coraria nepalensis; Coriandrum sativum; Curcuma longa; Datura metel syn alba; Daucus carota; Echinacea angustiflora and purpurea; Echinacea simulata; Echinacea pallida; Epimedium grandiflorum; Epimedium sagittatum; Epimedium sinense; Epilobium angustifolium; Erigeron Canadensis; Eugenia or Syzigium claviflorum; Fagara xanthox; Foeniculum vulgarel; Gardenia coronaria; Gaultheria trichophylla; Glycine max; Glycyrrhiza labra; Gossypium herbaceum; Heracleum sphondylium; Hypericum perforatum; Hypericum japonicum; Hyssopus officinalis; Jasminum officinale; Lithospermum erythrorhizon; Lonicerajaponica; Luffa luffa; Lycopus europaeus; Magnolia officinalis; Mallotus repandus; Mallotus philippinesis; Matricaria chamomil; Matricaria recutitia; Melissa parviflora; Melissa officinalis; Momordica balsamina; Momordica charantia; Narcissus tazetta; Narcissus pseudonarcissus; Oenthera rosea; Paeonia spec.; Papaver somniferum; Perilla frutescens; Phyllanthus niruri; Pinus koraicenis; Pinus parviflora; Piper nirgum; Plumeria rubra; Polyantha suberosa; Prunella vulgaris; Prunus bakariensis; Prunus amygdalus; Psoralea corylifolia; Randia dunatorum; Raphanus sativus; Rheum palmatum; Rhus coriaria; Rhus chinesis; Ricinus communis; Rosmarinus officinalis; Salvia miltiorhiza and officinalis; Sambucus ebulus; Saussurea lappa; Scilla griffithii; Scutellaria baicalensis baiealein; Sedum sediforme; Senecio scandens; Senecio aereus; Skimmia laureola; Solarium niporum; Swertia franchetiana; Terminalia chebula; Terminalia catappa; Terminalia alata; Thula occidentalis; Trapalaponica spec.; Trichosanthes dioica; Trichosanthes kirilowii; Urtica dioica; Viola yeodensis; Woodfordia fruticosa; Woodwardia spec. and Zanoxylum nitidum.

The herbal compositions described above used in combination with a chelator in a quantity sufficient to reduce a serum concentration of a bivalent metal in an amount of at least 25%.

Herbal Composition #2

In an especially preferred aspect, reverse transcriptase inhibitors (RTI) may be at least one of the following extracts from a plant that is known to have an antiviral effect, or an isolated or synthetically prepared compound that can be found in a plant known to have an antiviral effect. Especially contemplated plants include Abies webbiana; Acacia spec. Acacia Arabia; Agrimonia eupatoria; Ajuga decumbens; Allium cepa; Allium sativum; Aloe vera; Altemanthera philoxeroides or sessiles; Ammi maius; Andographis paniculata; Apium graveolens; Apium leptophyllum; Arachis hypogaea; Arctium lappa; Amebia euhcroma; Asparagus racemosus; Astragalus spinosus; Astragalus lentingosis swainsonine; Buchenavia capita; Bryonia cretica ssp. Dioica; Bryonia angustifolia; Camellia theifera; Camellia sinensis; Cedrela toona; Chrysanthemum morifolium; Coffea arabica; Coptis chinesis; Coptis teetoides; Coptis japonica; Coraria nepalensis; Coriandrum sativum; Curcuma longa; Datura metel syn alba; Daucus carota; Echinacea angustiflora and purpurea; Echinacea simulata; Echinacea pallida; Epimedium grandiflorum; Epimedium sagittatum; Epimedium sinense; Epilobium angustifolium; Erigeron Canadensis; Eugenia or Syzigium claviflorum; Fagara xanthox; Foeniculum vulgarel; Gardenia coronaria; Gaultheria trichophylla; Glycine max; Glycyrrhiza labra; Gossypium herbaceum; Heracleum sphondylium; Hypericum perforatum; Hypericum japonicum; Hyssopus officinalis; Jasminum officinale; Lithospermum erythrorhizon; Lonicera japonica; Luffa luffa; Lycopus europaeus; Magnolia officinalis; Mallotus repandus; Mallotus philippinesis; Matricaria chamomil; Matricaria recutitia; Melissa parviflora; Melissa officinalis; Momordica balsamina; Momordica charantia; Narcissus tazetta; Narcissus pseudonarcissus; Oenthera rosea; Paeonia spec.; Papaver somniferum; Perilla frutescens; Phyllanthus niruri; Pinus koraicenis; Pinus parviflora; Piper nirgum; Plumeria rubra; Polyantha suberosa; Prunella vulgaris; Prunus bakariensis; Prunus amygdalus; Psoralea corylifolia; Randia dunatorum; Raphanus sativus; Rheum palmatum; Rhus coriaria; Rhus chinesis; Ricinus communis; Rosmarinus officinalis; Salvia miltiorhiza and officinalis; Sambucus ebulus;Saussurea lappa; Scilla griffithii; Scutellaria baicalensis baiealein; Sedum sediforme; Senecio scandens; Senecio aereus; Skimmia laureola; Solarium niporum; Swertia franchetiana; Terminalia chebula; Terminalia catappa; Terminalia alata; Thula occidentalis; Trapalaponica spec.; Trichosanthes dioica; Trichosanthes kirilowii; Urtica dioica; Viola yeodensis; Woodfordia fruticosa; Woodwardia spec. Zanoxylum nitidum.

Alternatively it should be appreciated that RTIs other than herbal compositions are also appropriate, and such agents particularly include known and commercially available RTIs as indicated in Table 1.

| | Generic | Brand | Analogue |
|---|---|---|---|
| 3TC | lamivudine | Epivir/ | cytidine |
| ABC | abacavir | Ziagen | guanosine |
| AZT | zidovudine | Retrovir | thymidine |
| ddC | zalcitabine | HIVID | cytidine |
| ddI | didanosine | Videx | adenosine |
| d4T | stavudine | Zerit | thymidine |
| F-ddA | lodenosine | | adenosine |
| FTC | emtricitabine | Coviracil | cytidine |
| PMEA | adefovir | Preveon | adenosine |
| PMPA | tenofovir | adenosine | |

Herbal Composition #3

Particularly contemplated herbal compositions include Abies webbiana, Acacia spec., Acacia Arabia, Agrimonia eupatoria, Ajuga decumbens, Allium cepa, Allium sativum, Aloe vera, Altemanthera philoxeroides or sessiles, Ammi maius, Andographis paniculata, Apium graveolens, Apium leptophyllum, Arachis hypogaea, Arctium lappa, Amebia euhcroma, Asparagus racemosus, Astragalus spinosus, Astragalus lentingosis swainsonine, Buchenavia capita, Bryonia cretica ssp. Dioica, Bryonia angustifolia, Camellia theifera, Camellia sinensis, Cedrela toona, Chrysanthemum morifolium, Coffea arabica, Coptis chinesis, Coptis teetoides, Coptis japonica, Coraria nepalensis, Coriandrum sativum, Curcuma longa, Datura metel syn alba, Daucus carota, Echinacea angustiflora and purpurea, Echinacea simulata, Echinacea pallida, Epimedium grandiflorum, Epimedium sagittatum, Epimedium sinense, Epilobium angustifolium, Erigeron Canadensis, Eugenia or Syzigium claviflorum, Fagara xanthox, Foeniculum vulgarel, Gardenia coronaria, Gaultheria trichophylla, Glycine max, Glycyrrhiza labra, Gossypium herbaceum, Heracleum sphondylium, Hypericum perforatum, Hypericum japonicum, Hyssopus officinalis, Jasminum officinale, Lithospermum erythrorhizon, Lonicerajaponica, Luffa luffa, Lycopus europaeus, Magnolia officinalis, Mallotus repandus, Mallotus philippinesis, Matricaria chamomil, Matricaria recutitia, Melissa parviflora, Melissa officinalis, Momordica balsamina, Momordica charantia, Narcissus tazetta, Narcissus pseudonarcissus, Oenthera rosea, Paeonia spec., Papaver somniferum, Perilla frutescens, Phyllanthus niruri, Pinus koraicenis, Pinus parviflora, Piper nirgum, Plumeria rubra, Polyantha suberosa, Prunella vulgaris, Prunus bakariensis, Prunus amygdalus, Psoralea corylifolia, Randia dunatorum, Raphanus sativus, Rheum palmatum, Rhus coriaria, Rhus chinesis, Ricinus communis, Rosmarinus officinalis, Salvia miltiorhiza and officinalis, Sambucus ebulus, Saussurea lappa, Scilla griffithii, Scutellaria baicalensis baiealein, Sedum sediforme, Senecio scandens, Senecio aereus, Skimmia laureola, Solarium niporum, Swertia franchetiana, Terminalia chebula, Terminalia catappa, Terminalia alata, Thula occidentalis, Trapalaponica spec., Trichosanthes dioica, Trichosanthes kirilowii, Urtica dioica, Viola yeodensis, Woodfordia fruticosa, Woodwardia spec., and Zanoxylum nitidum. However, in alternative aspects many plants other than the above-listed plants are also contemplated. In fact, all plants are contemplated that exhibit antiviral activity.

Herbal Composition #4

Especially preferred antiviral compounds include herbal compositions and/or one or more isolated compounds (isolated from the plant or synthesized de novo) that are present in a herbal composition demonstrated to have an antiviral effect. Particularly suitable plants for contemplated extracts and isolated compounds include Abies webbiana; Acacia spec. Acacia Arabia; Agrimonia eupatoria; Ajuga decumbens; Allium cepa; Allium sativum; Aloe vera; Altemanthera philoxeroides or sessiles; Ammi maius; Andographis paniculata; Apium graveolens; Apium leptophyllum; Arachis hypogaea; Arctium lappa; Amebia euhcroma; Asparagus racemosus; Astragalus spinosus; Astragalus lentingosis swainsonine; Buchenavia capita; Bryonia cretica ssp. Dioica; Bryonia angustifolia; Camellia theifera; Camellia sinensis; Cedrela toona; Chrysanthemum morifolium; Coffea arabica; Coptis chinesis; Coptis teetoides; Coptis japonica; Coraria nepalensis; Coriandrum sativum; Curcuma longa; Datura metel syn alba; Daucus carota; Echinacea angustiflora and purpurea; Echinacea simulata; Echinacea pallida; Epimedium grandiflorum; Epimedium sagittatum; Epimedium sinense; Epilobium angustifolium; Erigeron Canadensis; Eugenia or Syzigium claviflorum; Fagara xanthox; Foeniculum vulgarel; Gardenia coronaria; Gaultheria trichophylla; Glycine max; Glycyrrhiza labra; Gossypium herbaceum; Heracleum sphondylium; Hypericum perforatum; Hypericum japonicum; Hyssopus officinalis; Jasminum officinale; Lithospermum erythrorhizon; Lonicera japonica; Luffa luffa; Lycopus europaeus; Magnolia officinalis; Mallotus repandus; Mallotus philippinesis; Matricaria chamomil; Matricaria recutitia; Melissa parviflora; Melissa officinalis; Momordica balsamina; Momordica charantia; Narcissus tazetta; Narcissus pseudonarcissus; Oenthera rosea; Paeonia spec.; Papaver somniferum; Perilla frutescens; Phyllanthus niruri; Pinus koraicenis; Pinus parviflora; Piper nirgum; Plumeria rubra; Polyantha suberosa; Prunella vulgaris; Prunus bakariensis; Prunus amygdalus; Psoralea corylifolia; Randia dunatorum; Raphanus sativus; Rheum palmatum; Rhus coriaria; Rhus chinesis; Ricinus communis; Rosmarinus officinalis; Salvia miltiorhiza and officinalis; Sambucus ebulus;Saussurea lappa; Scilla griffithii; Scutellaria baicalensis baiealein; Sedum sediforme; Senecio scandens; Senecio aereus; Skimmnia laureola; Solarium niporum; Swertia franchetiana; Terminalia chebula; Terminalia catappa; Terminalia alata; Thula occidentalis; Trapalaponica spec.; Trichosanthes dioica; Trichosanthes kirilowii; Urtica dioica; Viola yeodensis; Woodfordia fruticosa; Woodwardia spec. Zanoxylum nitidum;

Further preferred antiviral compounds include protease inhibitors (especially including Indinavir, Nelfinavir, Ritonavir, and Saquinavir), and reverse transcriptase inhibitors (especially including azidothymidine, dideoxyinosine, dideoxycytidine, deoxy-3′-thiacytidine, viramune, rescriptor, and 2′,3′-didehydro-3′-deoxythymidine). Where the antiviral compound comprises a nucleoside analog, particularly contemplated compounds include 1-beta-D-ribofuranosyl-1H-1,2,4-triazole-3-carboxamide (Ribavirin).

Herbal Composition #5

Contemplated compositions comprise one or more herbal compositions, which are prepared from various plants including Radix Glycyrrhizae; Folium Isatidis; Rhizoma Smilacis Glabrae; Flos Lonicerae; Rhizoma Atractylodis Macrocephalae; Fructus Ziziphi Jujubae; Ganoderma Lucidum Japonicum; Polygonum multiflorum Thunb; Radix Paeoniae Alba; Fructus Lycii; Rhizoma Polygonati; Radix Ophiopogonis; Poria; Herba Ecliptae; Fructus Schisandrae; Aconitum carmichaeli Debx; Radix Morindae Officinalis; Herba Epimedii; Fructus Comi; Radix Isatidis Radix Ginseng; Radix Angelicae Sinensis; Radix Astragali seu Hedysari; Fructus Ligustri Lucidi; Radix Sophorae Flavescentis; Radix Trichosanthis; Herba Agrimoniae; Ganoderma Lucidum seu Japonicum; Radix Rehmanniae; Cordyceps; Borneolum Syntheticum; Cordyceps Sinensis; Olenlandia Diffusae; Natural Indigo; Polyporous Umbellatus; Astragalus Membranaceous; Panax Ginseng; Solanum Nigrum L; Pogostemon Cablin; Atractylodes Macrocephalae; Trichosanthis Radix; Clematidis Radix; Margarita; Ligustrum Lucidum AIT; and Glycyrrhizae Radix.

In a preferred embodiment, the herbal composition prepared from a plant selected from the group consisting of: Radix Ginseng; Radix Angelicae Sinensis; Radix Astragali seu Hedysari; Fructus Ligustri Lucidi; Radix Sophorae Flavescentis; Radix Trichosanthis; Herba Agrimoniae; Ganoderma Lucidum seu Japonicum; Radix Rehmanniae; Cordyceps; and Borneolum Syntheticum. Alternatively, the herbal composition may comprise Cordyceps Sinensis; Olenlandia Diffusae; Natural Indigo; Polyporous Umbellatus; Astragalus Membranaceous; Panax Ginseng; Solanum Nigrum L; Pogostemon Cablin; Atractylodes Macrocephalae; Trichosanthis Radix; Clematidis Radix; Margarita; Ligustrum Lucidum AIT; and Glycyrrhizae Radix.

Examples of Preparing Herbal Composition #5

Contemplated compositions may be produced using a variety of methods. However, it is preferred that the following method be used:

First, the extract is prepared. Radix Angelicae Sinensis; Radix Astragali seu Hedysari; Fructus Ligustri Lucidi; Radix Sophorae Flavescentis; Radix Trichosanthis; Herba Agrimoniae; and Ganoderma Lucidum seu Japonicum are cleaned and dried. Then they are placed into an extracting tank. Four parts water is added to 1 part herbal composition. The mixture is then boiled for approximately two hours. The liquid is extracted, producing a first extractant and a first supernatant. The supernatant is poured off. Next, water is again added, now at a ratio of 3 parts water to 1 part supernatant. The mixture is then boiled for approximately 1 hour. The liquid is then extracted for the second time, producing a second extractant and a second supernatant. Then, the first and second extractants are mixed together, filtered, and concentrated.

Second, the powder is prepared. Radix Ginseng, Radix Rehmanniae, and Cordyceps are cleaned. They are then dried using a vacuum cold drying process wherein the temperature is approximately 48 degrees Celsius. Next, the dried plant material is ground into a powder.

Third, the extract and the powder are mixed together well. This mixture is further dried using the vacuum cold drying process. After drying, the mixture is ground to a powder and sifted. Finally, Calculus Bovis and Borneolum Syntheticum are added to the mixture, and mixed well.

Lastly, the powder is put into capsules, with each capsule containing approximately 0.25 grams of powder.

It should be appreciated that the approximate raw material to end material ratio for the following ingredients: Radix Angelicae Sinensis; Radix Astragali seu Hedysari; Fructus Ligustri Lucidi; Radix Sophorae Flavescentis; Radix Trichosanthis; Herba Agrimoniae; and Ganoderma Lucidum seu Japonicum are 6:1 while the other ingredients (Radix Ginseng, Calculus Bovis, Radix Rehmanniae, Cordyceps; and Bomeolum Syntheticum) are in a ratio of 1:1.

The following table (Table 1) depicts the ingredients, genus, species, and percentage of each ingredient that is present within the composition.

TABLE 1

| | Ingredients | Genus | species | % of total |
|---|---|---|---|---|
| 1 | Radix Ginseng | Panax | ginseng | 5% |
| 2 | Radix Angelicae | Angelica | sinensis | 15% |
| | Sinensis | | | |
| 3 | Radix Astragali seu | Astragalus | membranaceus | 15% |
| | Hedysari | | | |
| 4 | Fructus Ligustri | Ligustrum | lucidum | 10% |
| | Lucidi | | | |
| 5 | Radix Sophorae | Sophora | flavescens | 10% |
| | Flavescentis | | | |
| 6 | Radix Trichosanthis | Trichosanthes | kirilowii | 15% |
| 7 | Calculus Bovis | Bos | taurus | 1% |
| | | | domesticus | |
| 8 | Herba Agrimoniae | Agrimonia | pilosa | 10% |
| 9 | Ganoderma Lucidum | Ganoderma | lucidum | 5% |
| | seu Japonicum | | | |
| 10 | Radix Rehmanniae | Rehmannia | glutinosa | 10% |
| 11 | Cordyceps | Cordyceps | sinensis | 3% |
| 12 | Borneolum | Dryobalanops | aromatica | 1% |
| | Syntheticum | | | |

Similarly, the following table (Table 2) depicts the number of kilograms of the raw ingredients that will result in 1 kilogram of the finished product:

TABLE 2

| Ingredients | Kilo |
|---|---|
| Radix Ginseng | 0.15 |
| Radix Angelicae Sinensis | 0.45 |
| Radix Astragali seu Hedysari | 0.45 |
| Fructus Ligustri Lucidi | 0.3 |
| Radix Sophorae Flavescentis | 0.3 |
| Radix Trichosanthis | 0.45 |
| Calculus Bovis | 0.03 |
| Herba Agrimoniae | 0.3 |
| Ganoderma Lucidum seu Japonicum | 0.15 |
| Radix Rehmanniae | 0.3 |
| Cordyceps | 0.09 |
| Borneolum Syntheticum | 0.03 |

In a further preferred embodiment, for example, the method above may be followed using the following ingredients in their respective amounts (See Table 3):

TABLE 3

| | |
|---|---|
| Cordyceps sinensis | 1150 mg |
| Olenlandia diffusae | 425 mg |
| Natural Indigo | 425 mg |
| Polyporus umbellatus | 270 mg |
| Astragalus membranaceous | 255 mg |
| Panax ginseng | 255 mg |
| Solanum nigrum L | 140 mg |
| Pogostemon cablin | 140 mg |
| Atractylodes macrocephalae | 125 mg |
| Trichosanthis radix | 125 mg |
| Clematidis radix | 125 mg |
| Margarita (Pearl from oyster in powder form) | 65 mg |
| Ligustrum lucidum AIT. | 65 mg |
| Glycyrrhizae radix | 65 mg |

It should be appreciated that the percentages and amounts represented here may vary depending on various factors including pureness of herbal compositions available, severity of symptoms, weight and age of patient, and various other factors.

Although this example showed preparation of Herbal Composition #5, the same or similar preparations may be employed to prepare other compositions herein disclosed.

Chelators

With respect to the chelator it is generally contemplated that all chelating agents are suitable so long as they are physiologically acceptable in the concentration and route of administration used. Contemplated chelators for use in conjunction with the teachings presented herein may be used so long as such chelators (a) reduce serum concentration of a bivalent metal (e.g. Ca²⁺ and Mg²⁺) in an amount of at least 25%, and (b) are at least partially effective to promote viral disintegration at an administered dosage. Particularly contemplated bivalent metals include Ca²⁺ and Mg²⁺. Particularly preferred chelators include 1,2-Bis(2-aminophenoxy)ethane-N,N,N′,N′-tetraacetic acid, Ethylenebis(oxyethylenenitrilo)tetraacetic acid, 1,2-bis(2-aminophenoxy)ethane-N,N′,N′-tetraacetic acid tetrakis(acetoxymethyl ester), trans-1,2-diaminocyclohexane-tetraacetic acid, and diethyllenetriamine-pentaacetic acid, trimethylaminetricarboxylic acid, poly(aspartic acid), and poly(glutamic acid), ethylenediamine-N,N,N′, N′,-tetraacetic acid, and EGTA.

The inventors further contemplate that treatment of a viral infection can be significantly improved by administration of a chelator in a time-release formulation. Furthermore, the inventors contemplate that the chelator is co-administered in a time-release formulation with a second or further agent with antiviral effect (which may be administered following a conventional protocol or in a second time release formulation). Contemplated viruses include retroviruses (e.g., HV, HCV), ssDNA and dsDNA.

There are numerous methods of preparing a time release formulation known in the art, all of which are contemplated suitable for use in conjunction with the teachings herein. However, particularly contemplated time release formulations include ion exchange resins, encapsulations with acid or base resistant coatings, compacting the formulation to control solvation, slow-melting carriers, enzyme-degradable carriers, etc.

Depending on the amount chelator in contemplated compositions, it is contemplated that the viral titer in the serum of a patient infected with the virus will decrease at least 10% for at least 4 hours, more preferably at least 25% for at least 6 hours, and most preferably at least 40% for at least 8 hours after administration of a single dose of contemplated compounds.

Bile Products

“Bile products” as used herein means any product containing or derived from bile, including, for example bile, bile salts, bile stones, gall bladder stones, and cholic acids from any animal.

However, it is preferred that bile products comprise calculus Bovis, also sometimes called cow bezoar. Calculus Bovis is generally the gall stone or bile of an ox, water buffalo, or pig, which is often dried and made into a powder for pills.

Calculus Bovis is generally used as an anti-inflammatory, antipyretic, and/or antibacterial agent. For example, it may be used for high fever with accompanying delirium and convulsion. It may also be used for chronic sore throat or internal abscesses that have ruptured.

Calculus Bovis is typically expensive and primarily used in Chinese medicine. The combination of niu huang with rhinoceros or water buffalo horn may be used in the treatment of Legionnaire's disease, meningitis, and encephalitis. A typical dosage is 0.15-0.3 grams, but any non-toxic dosage may be used if effective.

Cholic acids are naturally-occurring chenodeoxycholic acid and its synthetic derivative, urosdeoxycholic acid. Both are often used in the oral dissolution of cholesterol gall stones. Ursodeoxycholic acid may also be used in primary biliary cirrhosis and primary sclerosing cholangitis.

Results

Symptomatic Relief

Methods of improving quality of life in an AIDS patient are contemplated wherein a patient is diagnosed as having or being infected with AIDS, and a sufficient amount of the composition is administered to the patient in a sufficient quantity to improve the quality of life of the A/DS patient. The quality of life is measured on a scale of 1 to 4, with 1 being no improvement and 4 being great improvement, and each of at least three of the following: sleep, energy level, discomfort, appetite, and mental clarity, are increased by a scaled rate of at least 2.

Furthermore, suitable compositions will improve symptoms associated with various diseases, including AIDS. Contemplated symptoms include pain (peripheral neuropathy); fever, cough, and other cold/flu symptoms; night sweats; diarrhea, nausea, and other indigestion symptoms; lymph swelling or other immunological symptoms; weight loss and loss of appetite; candida in the mouth; secondary bacterial and/or viral infections; elevated liver enzymes; reduction in central nervous system and brain function; depression; and overall reduced immunity. Especially contemplated symptoms include difficulty in sleeping or insomnia, reduced energy level and lethargy, increased discomfort, loss of appetite, and reduction in mental clarity.

With respect to the administration of contemplated compositions, it should be recognized that various protocols are suitable, and especially contemplated protocols include substantially simultaneous administration of the chelator (e.g., coadministration in a single tablet), or administration of the chelator (or herbal composition) while there is a measurable concentration of the herbal composition (or chelator) in the patient. For example, it is contemplated that suitable herbal compositions may be orally administered, while the chelator is parenterally administered (e.g., via injection or mucosal presentation). In preferred embodiments, the extract is administered in one vial two times a day, or even more preferably, two vials a day.

Consequently, the dosage and formulation of contemplated herbal compositions and chelators may vary substantially. However, it is preferred that the extract is administered in approved and/or known dosages and formulations. Similarly, it is preferred that dosages and formulations of appropriate chelators are identical or similar to those known in the art.

Further contemplated methods include marketing the composition for use in alleviating symptom associated with a disease, preferably AIDS. The preferred method is to package the composition with labeling identifying the composition as being effective in alleviating or improving at least some symptoms experienced by AIDS patients. All manners of packaging the composition are suitable, especially placing the composition into bottles, boxes, and blister packets.

Clinical Studies

10 AIDS patients were studied from April to September, 1993 with the following results:
(2) Patients were chosen according to the following criteria
   (a) Patients tested positive to HIV using ELISA and/or Western Blot.
   (b) Patients' immune function was abnormal (i.e. the T4 to T8 ratio was under 1, the number of T4 cells was under 400/mm³)
   (c) The average age of the patient was above 7 years.
   (d) No pregnant women were chosen.
   (e) The patient agreed voluntarily to use traditional Chinese medicine techniques and treatments.
(3) The patients were observed according to the following:
   (a) Patients were required to take an ELISA test to indicate the presence of the HIV virus.
   (b) Patients underwent testing with monoclonal antibody reagent and flow cytometry (FACSCAN) every three months before or after treatment to calculate the rate of T4 to T8, and the quantity of T4 (in cubic centimeters) as the index to measure the immune function of treated patients.

(c) Complete diagnostic records were recorded, including physical sign or appearance, appearance of the tongue, and pulse condition. The following symbols were used: evident: (+++); general (++); slight (+); fluctuant ±); and negative (−) to label the symptoms and convert them into a raw score for statistical use. The conversion was as follows (See Table 4):

TABLE 4

| Score | Symbol |
|---|---|
| 6 | +++ |
| 4 | ++ |
| 2 | + |
| 1 | ± |
| 0 | − |

(4) Two capsules of the composition described in the Examples (See Table 1) were given three times a day over a course of 3 months
(5) Curative Effect:
   (a) Method: A comprehensive analysis of the curative effect according to the immune state and clinical symptoms before or after treatment were studied and recorded. The above mentioned scores were used to show the clinical symptoms of each patient. The ratio of T₄ to T₈ and number T₄ cells was observed and used as criteria to measure the state of the immune system: if both of the items increase or either of them increase without a change in the other, there is an improvement in immune function; if both of the items decrease or either of them decrease without a change in the other, the immune system worsens; if neither of the items change, then there is no change in the immune system; if one of them increases while the other decreases, the immune system is judged according to the increase or decrease in the quantity of T₄ cells.
   (b) Criteria for curative effect: The various criteria are categorized as recovery, evident effect, with effect and no effect are divided according to the stipulate of scientific research plan.
   (c) Recovery: if serum antibody is negative (with the method of ELISA and WB) and the testing of antigen P24 and PCR is also negative, the immune function recovers to normal (ratio of T₄ to T₈>1 and the number of T₄ cells are beyond 800/mm³). There are no symptoms, no physical signs and no opportunity infections.
   (d) Evident effect: If Antigen HIV appears negative or positive, the immune function improves dramatically (ratio of T₄ to T₈>1, but the number of T₄ cell is 400/mm³), the opportunity infection is basically removed, and the symptoms and physical signs recover fundamentally back to normal.
   (e) With effect: If Antibody HIV appears positive, the immune function is improving (ratio of T₄ to T₈>0.2, the quantity of T₄ cells is beyond 40/mm³). Opportunity infection is also improving, and the symptoms and physical signs are relieved.
   (f) No effect: If there are no dramatic changes on antibody HIV including index of immune function, there is a decrease of immune function in the treatment.
(6) Clinical observation

General Clinical File

Total number of patients and cases is 10: 5 men and 5 women. The ratio is 1:1.

Average age is about 31.33, with the oldest being 35, and the youngest being 25.

Period of the disease: According to the CDC, the 10 cases in this class all have HIV symptoms, 4 of which have ARC and 6 of which are AIDS.
(7) Results of treatment

Change of weight: The weight of patients in the treatment class are relatively stable, with the fluctuation being approximately 2 kg.

Clinical symptoms: Observed the change of 11 items such as tiredness, fever, cough, shortness of breath, achiness in the chest, diarrhea, anorexia, eruption, night sweat, lymphadenectasis and stomatocace before and after the treatment. The 10 items all appear to be in good condition in different degrees and the average score 4.6 (2-6 score) is relieved.

Change of Immune Function:
a. The rate of T4 to T8 and the number of T4 cells: The ratio of T4 to T8 of the 10 patients before and after the treatment shows 3 patients who have increased ratios, while the other 7 show no evident change. The numbers of T4 cells of the 3 patients increased.
b. Evaluating the state of immune function: According to the study, the immune function is divided into three conditions after treatment such improvement, no change and worse. In this study, 3 cases improved, 3 cases showed no change and 4 cases became worse.
c. Curative effect: According to the criteria of curative effect 6 cases are effective and 4 cases are ineffective in this study.
d. Side effects of medicine: 6 cases of the class showed side effects in varying degrees, namely 2 cases felt nauseated, 2 cases felt anorexia, 1 case felt diarrhea and 1 case felt tired and itchy. However, all the cases continued to take the medicine in the prescribed quantity.

In conclusion, with respect to all the patients in the class, 4 cases were ARC and the other 6 cases were AIDS. The conditions of the aimed patients were critical. In the treatment, most of the symptoms of the cases showed improvement after taking the medicine. A score of 4.67 (2-6 score) was the average. 3 cases immune function improved. In another 3 cases, the number of T4 cells increased. Thus, 6 cases were effective, and 4 cases were ineffective. The side effects were common, but most of them were slight and all the cases were able to take the medicine continuously.

In a different study, patients were given combination therapy with EGTA as a suppository and a composition to relieve symptoms suffered by AIDS patients. Clinical data was reported as follows:

CLINICAL DATA

| | DATE: | | | | |
|---|---|---|---|---|---|
| PATIENT 1 | May 2002 | September 2002 | October 2002 | December 2002 | |
| HIV-1 RNA | 735 | 67 | 1,607* | <50 | IMPROVED |
| CD4 | 675 | 537 | 896 | 698 | NO CHANGE |
| CD8 | 978 | 799 | 1008 | 1014 | NO CHANGE |

| PATIENT 2 | June 2002 | September 2002 | December 2002 | |
|---|---|---|---|---|
| HIV-1 RNA | <50 | SAME | SAME | |
| CD4 | 423 | 421 | 461 | IMPROVED |
| CD8 | 1952 | 1045 | 1050 | IMPROVED |
| AST/ALT | 100/111 | 116/200 | 50/59 | IMPROVED |

| OVERALL QUALITY OF LIFE DATA | PT 1 | PT 2 |
|---|---|---|
| SLEEP IMPROVEMENT | 2 | 2/3 |
| INCREASE IN ENERGY LEVEL | 4 | 4 |
| DECREASE IN DISCOMFORT | 3 | 3/4 |
| IMPROVEMENT IN APPETITE | 3 | 3 |
| IMPROVEMENT IN MENTAL CLARITY | 4 | 4 |
| Average: | 3.2 | 3.4 |

KEY:
1-NONE
2-SLIGHT
3-MODERATE
4-GREAT
Overall improvement-moderate to great

Thus, specific embodiments and applications of antiviral treatments using a herbal composition in combination with (a) a chelator and/or (b) a bile product have been disclosed. It should be apparent, however, to those skilled in the art that many more modifications besides those already described are possible without departing from the inventive concepts herein. The inventive subject matter, therefore, is not to be restricted except in the spirit of the appended contemplated claims. Moreover, in interpreting both the specification and the contemplated claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A combination comprising an herbal composition and a chelator in sufficient quantities to improve at least one of the following when administered to a person having AIDS or ARC (AIDS related complex): sleep, energy level, discomfort, appetite, or mental clarity.

2. The combination of claim 1 further comprising a bile product

3. The combination of claim 2 wherein the bile product is calculus bovis.

4. The combination of claim 1 wherein the herbal composition is prepared from a plant selected from the group consisting of Radix Ginseng; Radix Angelicae Sinensis; Radix Astragali seu Hedysari; Fructus Ligustri Lucidi; Radix Sophorae Flavescentis; Radix Trichosanthis; Herba Agrimoniae; Ganoderma Lucidum seu Japonicum; Radix Rehmanniae; Cordyceps; Bomeolum Syntheticum; Cordyceps Sinensis; Olenlandia Diffusae; Natural Indigo; Polyporous Umbellatus; Astragalus Membranaceous; Panax Ginseng; Solanum Nigrum L; Pogostemon Cablin; Atractylodes Macrocephalae; Trichosanthis Radix; Clematidis Radix; Margarita; Ligustrum Lucidum AIT; and Glycyrrhizae Radix.

5. The combination of claim 1 wherein the herbal composition is prepared from Radix Sophorae Flavescentis.

6. The combination of claim 1 wherein the herbal composition is prepared from Clematidis Radix.

7. The combination of claim 1 wherein the chelator is selected from the group consisting of 1,2-Bis(2-aminophenoxy)ethane-N,N,N′,N′-tetraacetic acid, Ethylenebis(oxy-ethylenenitrilo)tetraacetic acid, 1,2-bis(2-aminophenoxy)ethane-N,N,N′,N′-tetraacetic acid tetrakis(acetoxymethyl ester), trans-1,2-diaminocyclohexane-tetraacetic acid, and diethyl-lenetriamine-pentaacetic acid.

8. The combination of claim 7, wherein the chelator is 1,2-Bis(2-aminophenoxy)ethane-N,N,N′,N′-tetraacetic acid.

9. The combination of claim 7, wherein the chelator is trans-1,2-diaminocyclohexane-tetraaceitic acid.

10. The combination of claim 1 wherein the chelator is selected from the group consisting of tri-methylaminetricarboxylic acid, poly(aspartic acid), and poly(glutamic acid).

11. A method of improving a quality of life in a person having AIDS or ARC, comprising:
determine that a person is infected with AIDS; and
administering a sufficient amount of the composition of claim 1 to the person;
wherein the quality of life comprises at least one of sleep, energy level, discomfort, appetite, and mental clarity.

12. The method of claim 11 wherein the quality of life is measured on a scale of 1 to 4, with 1 being no improvement and 4 being great improvement, and each of at least three of the following: sleep, energy level, discomfort, appetite, and mental clarity, are increased by a scaled rate of at least 2.

13. A method of marketing the composition of claim 1 for use in alleviating a symptom associated with a disease.

14. The method of claim 13 wherein the disease is AIDS.

15. The method of claim 13 wherein the symptom is energy level.

16. The method of claim 13 wherein the symptom is discomfort.

17. A composition for use in alleviating a symptom associated with an HIV infection comprising:
a herbal composition prepared from a plant selected from the group consisting of: Radix Ginseng; Radix Angelicae Sinensis; Radix Astragali seu Hedysari; Fructus Ligustri Lucidi; Radix Sophorae Flavescentis; Radix Trichosanthis; Herba Agrimoniae; Ganoderma Lucidum seu Japonicum; Radix Rehmanniae; Cordyceps; Borneolum Syntheticum; and
a bile product.

18. The composition of claim 17, further comprising 5% Radix Ginseng, 15% Radix Angelicae Sinensis, 15% Radix Astragali seu Hedysari, 10% Fructus Ligustri Lucidi, 10% Radix Sophorae Flavescentis, 15% Radix Trichosanthis, 1% Calculus Bovis, 10% Herba Agrimoniae, 5% Ganoderma Lucidum seu Japonicum, 10% Radix Rehmanniae, 3% Cordyceps, and 1% Borneolum Syntheticum.

19. The composition of claim 17, further comprising a chelator.
